(19) United States
(12) Patent Application Publication
(10) Pub. No.: US 2022/0046326 A1
(43) Pub. Date: Feb. 10, 2022

(54) MEDICAL VIDEO DISTRIBUTION SYSTEM AND MEDICAL VIDEO DISTRIBUTION METHOD

(71) Applicant: Medi Plus Inc., Tokyo (JP)

(72) Inventors: Naoya Sugano, Tokyo (JP); Minsu Kwon, Tokyo (JP)

(73) Assignee: Medi Plus Inc., Tokyo (JP)

(21) Appl. No.: 17/312,643

(22) PCT Filed: Dec. 2, 2019
(86) PCT No.: PCT/JP2019/047060

§ 371 Date: Jun. 10, 2021

(30) Foreign Application Priority Data
Dec. 17, 2018 (JP) 2018-235455

(51) Int. Cl. H04N 21/485; H04N 21/231; H04N 21/234; H04N 21/2365; H04N 21/431

(52) U.S. Cl. CPC H04N 21/4858; H04N 21/231; H04N 21/4312; H04N 21/2365; H04N 21/234

(57) ABSTRACT

Provided are a medical video distribution system and a medical video distribution method, capable of stably streaming video output from medical devices. A server device executes an editing process (step S206) editing video data output from origin(s) designated by step S201, on a basis of correspondence designated by step S205, in display area(s) contained in a layout designated by step S203; and a first distribution process (step S209) streaming the edited video data towards display terminal(s), and, when there are plurality of designated origins, synthesizes in the editing process a plurality of video data output from the origins to produce a single synthesized video data having a data volume nearly equal to that of the video data output from a single origin.

### TECHNICAL FIELD

The present invention relates to a medical video distribution system and a medical video distribution method.

### BACKGROUND ART

In recent years, medical surgery has been usually conducted, while distributing medical video, so that surgical process can be checked not only on monitors installed within an operating room, but also on monitors installed outside of the operating room, on which video data that contains video of surgical field, measured results of vital information and so forth are displayed.

Patent Literature 1 below is exemplified as one of prior arts of this sort of medical video distribution.

Patent Literature 1 discloses a technology by which a partial data is extracted from a plurality of inspection data obtained by contrast radiography, intravascular sonography and so forth, within a range corresponded to a certain title of treatment, referring to a procedure log that correlates the title of treatment given to a patient and treatment time, and then synchronously display the extracted partial data.

### CITATION LIST

### Patent Literature

[Patent Literature 1]JP-2008-301984 A

### SUMMARY OF THE INVENTION

### Technical Problem

According to the technology disclosed in Patent Literature 1, such as a system that sends images from a server device to a computer terminal to thereby display a plurality of images at the same time on a single display device, any trial to transfer the individual images from the server device to the computer terminal by streaming will encounter a problem that the larger the number of images, the larger the traffic, and more difficult the stable streaming would be.

The present invention was arrived at in consideration of the aforementioned problem, and is to provide a medical video distribution system and a medical video distribution method that enable stable streaming of the videos output from the medical devices.

### Solution to Problem

According to the present invention, there is provided a medical video distribution system that includes: a plurality of medical devices that output video data; a server device that stores the video data output from the medical devices; and one or a plurality of display terminals that receive, and then play, the video data received from the sever device by way of streaming, the display terminal being designed to execute: an origin designation process designating one or a plurality of origins of the video data from among the plurality of medical devices; a layout designation process designating a layout of the video data to be displayed on the display terminal(s); and an correspondence designation process freely designating to which display area(s) the origin(s) having been designated by the origin designation process to be assigned, out of one or a plurality of display areas contained in the layout designated by the layout designation process, in response to an user's operation, and, the server device being designed to execute: an editing process editing the video data output from the origin(s) designated by the origin designation process, on a basis of the correspondence designated by the correspondence designation process, in the display area(s) contained in the layout designated by the layout designation process; and a first distribution process streaming the video data having been edited by the editing process towards the display terminal(s), and, when there are plurality of origins designated by the origin designation process, the server device being designed to synthesize, in the editing process, a plurality of video data output from the origins to produce a single synthesized video data having a data volume nearly equal to that of the video data output from a single origin, and to stream, in the first distribution process, the single synthesized video data towards the display terminal(s).

According to the present invention, there is also provided a medical video distribution method using: a plurality of medical devices that output video data; a server device that stores the video data output from the medical devices; and one or a plurality of display terminals that receive, and then play, the video data received from the sever device by way of streaming, the method includes: a first step designating one or a plurality of origins of the video data from among the plurality of medical devices; a second step designating a layout of the video data to be displayed on the display terminal(s); a third step freely designating to which display area(s) the origin(s) having been designated by the first step to be correlated, out of one or a plurality of display areas contained in the layout designated by the second step, in response to an user's operation; a fourth step editing the video data output from the origin(s) designated by the first step, on a basis of the correspondence designated by the third step, in the display area(s) contained in the layout designated by the second step; and a fifth step streaming the video data having been edited by the fourth step towards the display terminal(s), the method being designed, when there are plurality of origins designated by the first step, to synthesize in the fourth step a plurality of video data output from the origins to produce a single synthesized video data having a data volume nearly equal to that of the video data output from a single origin, and to stream, in the fifth step, the single synthesized video data towards the display terminal(s).

Upon selection of a plurality of origins, the present invention synthesizes, before streaming, the individual video data to produce video data (a single synthesized video data) having a data volume nearly equal to that of the video data output from a single origin, thus successfully stabilizing the traffic of the system as a whole.

### Advantageous Effects of Invention

According to the present invention, there are provided a medical video distribution system and a medical video distribution method that enable stable streaming of the videos output from the medical devices.

### BRIEF DESCRIPTION OF DRAWINGS

FIG. 1 is a drawing illustrating a medical video distribution system of an embodiment.

FIG. 2 is a sequence chart illustrating action procedures between a display terminal and a server device.

FIG. 3 is a drawing illustrating operation methods and screen shots in layout designation and correspondence designation.

FIG. 4 is a drawing illustrating a screen shot of a video streamed on the display terminal.

FIG. 5 is a sequence chart illustrating action procedures between a display terminal and a server device.

FIG. 6 is a drawing illustrating operation methods and screen shots of the display terminal in layout designation and correspondence designation.

FIG. 7 is a drawing illustrating screen shots of the videos streamed on the display terminal and operation methods on the display terminal.

### DESCRIPTION OF EMBODIMENTS

Embodiments of the present invention will be explained below referring to the attached drawings. Note that all similar constituents in all drawings will be given same reference signs, so as to suitably avoid redundant explanation.

### <Individual Constituents in Medical Video Distribution System 100>

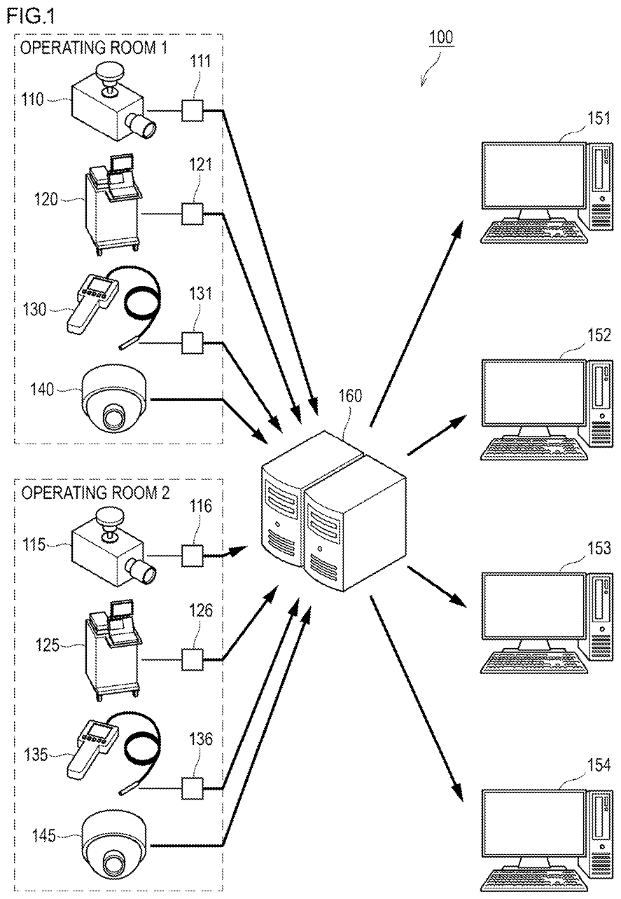

FIG. 1 is a drawing illustrating a medical video distribution system of an embodiment (referred to as a medical video distribution system 100, hereinafter).

Note that the arrows illustrated in FIG. 1 indicate origins and destinations of video data, sent and received among the individual constituents. Hence any information or data other than the video data does not always necessarily coincide with the meaning indicated by these arrows.

The medical video distribution system 100 has a plurality of medical devices (surgical field cameras 110 and 115, biomonitors 120 and 125, endoscope cameras 130 and 135, panorama cameras 140 and 145), a plurality of display terminals 151 to 154, and a server device 160.

The surgical field cameras 110 and 115, the biomonitors 120 and 125, the endoscope cameras 130 and 135, and the panorama cameras 140 and 145 individually have a function of outputting video data towards the outsides thereof, and correspond to the plurality of medical devices in the context of the present invention.

The surgical field camera 110 is a camera installed in a first operating room (referred to as an operating room 1, hereinafter), and the surgical field camera 115 is a camera installed in a second operating room (referred to as an operating room 2, hereinafter), both can be used for the purpose of shooting a surgical field during operation.

The biomonitor 120 is a measuring instrument installed in the operating room 1, and the biomonitor 125 is a measuring instrument installed in the operating room 2, both can measure so-called vital signs, and can display results of measurement in the form of video. The vital sign includes statuses regarding blood pressure, heart rate, respiration rate, body temperature and so forth.

The endoscope camera 130 is a device installed in the operating room 1, the endoscope camera 135 is a device installed in the operating room 2, both can be used for the purpose of enabling in vivo observation and certain treatment of a subject, by inserting a tube with a camera and a surgical tool equipped to the distal end.

The panorama camera 140 is a camera installed in the operating room 1, and the panorama camera 145 is a camera installed in the operating room 2. Videos shot by these cameras can be used for the purpose of capturing a panorama of each operating room.

Note that the medical device enumerated above are merely examples of the articles composing the plurality of medical devices in the present invention, so that any medical device having not been enumerated above may be used in the present invention, or any medical device having been enumerated above may be left unused in the present invention. Moreover, the articles composing the medical devices in the present invention are not always necessarily devices which are applicable solely to medical purposes, but may be devices which are applicable to other purposes.

Although having explained the above-enumerated cameras (surgical field camera and panorama camera) as articles for shooting the surgical field and the panoramas of the operating rooms, there are no special limitation on sites of shooting, and mode of shooting of the cameras (normal-angle shooting or wide-angle shooting, etc.).

The server device 160 is a device that receives and stores the video data output from the aforementioned various medical devices, and streams the stored video data in response to a playback request accepted from the display terminals 151 to 154, which corresponds to the server device in the context of the present invention.

The server device 160 uses an internet protocol (IP) address both when receiving the video data output from the medical devices and when streaming the stored video data, in order to specify the other party. Some medical devices, having no IP address, are connected to the server device 160 individually via an IP converter to establish communication. Explaining now referring to the exemplary case of this embodiment, the surgical field cameras 110 and 115 are connected via an IP converter 111, the biomonitor 120 is connected via an IP converter 121, the biomonitor 125 is connected via an IP converter 126, the endoscope camera 130 is connected via an IP converter 131, and the endoscope camera 135 is connected via an IP converter 136 to the server device 160. In contrast, the panorama cameras 140 and 145 and the display terminals 151 to 154 are connected to the server device 160 in a communicable manner without being mediated by the IP converters, since each of them employs an IP camera to which an IP address is individually assigned.

Note now that the server device 160, although illustrated in FIG. 1 as a twin-computer device, may alternatively be constructed by a single computer device, or may be constructed by three or more computer devices.

Also note that the streaming in the context of this embodiment include livestreaming (a distribution system concurrently enabling downloading of the video data to the server device 160, and streaming by the server device 160 towards the display terminals 151 to 154).

Also note that, although the panorama cameras 140 and 145 were given as the IP cameras in this embodiment, other types of camera (for example, the surgical field cameras 110 and 115) may be given as the IP cameras, or, the IP camera does not have to be employed.

The display terminals 151 to 154 are computer devices that stream the video data downloaded from the server device 160, and correspond to the display terminals in the context of the present invention.

The display terminals 151 to 154 has installed thereon application software (viewer) for reproducing the video data to be streamed. The viewer installed on the display terminals 151 to 154 is not always necessarily embodied by application software specialized to the present invention, instead may be embodied by general-purpose application software (internet browser, for example), or improvement or modification thereof.

Note that all processes explained below, executable on the display terminal 151, are understood to be similarly executable also on the other display terminals (display terminals 152 to 154).

<Action Procedures between Display Terminal 151 and Server Device 160>

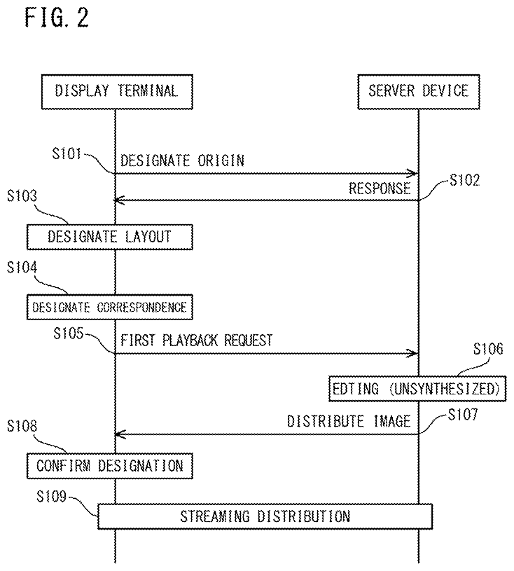

Next, the action procedures between the display terminal 151 and the server device 160 will be explained referring to FIGS. 2 to 7.

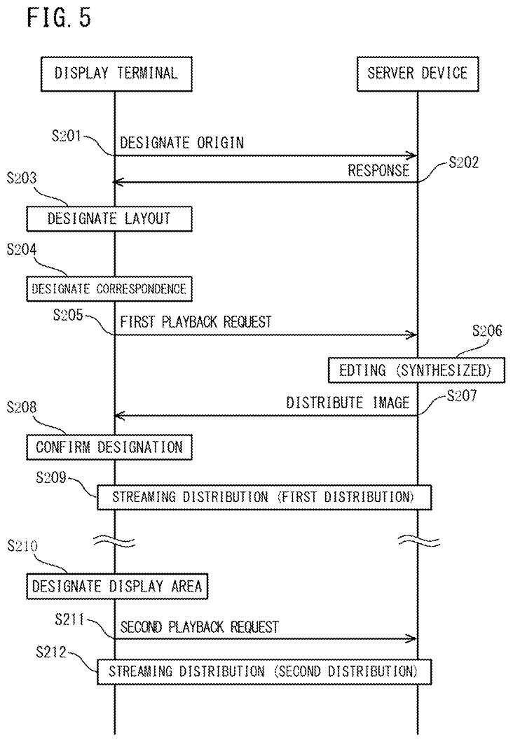

FIGS. 2 and 5 are sequence charts illustrating the action procedures between the display terminal 151 and the server device 160. Note that the processes illustrated in the sequence charts are merely exemplary ones necessary for explaining the present invention, allowing addition of unillustrated processes or omission of the illustrated processes, within a scope that the purpose of the present invention will be attained.

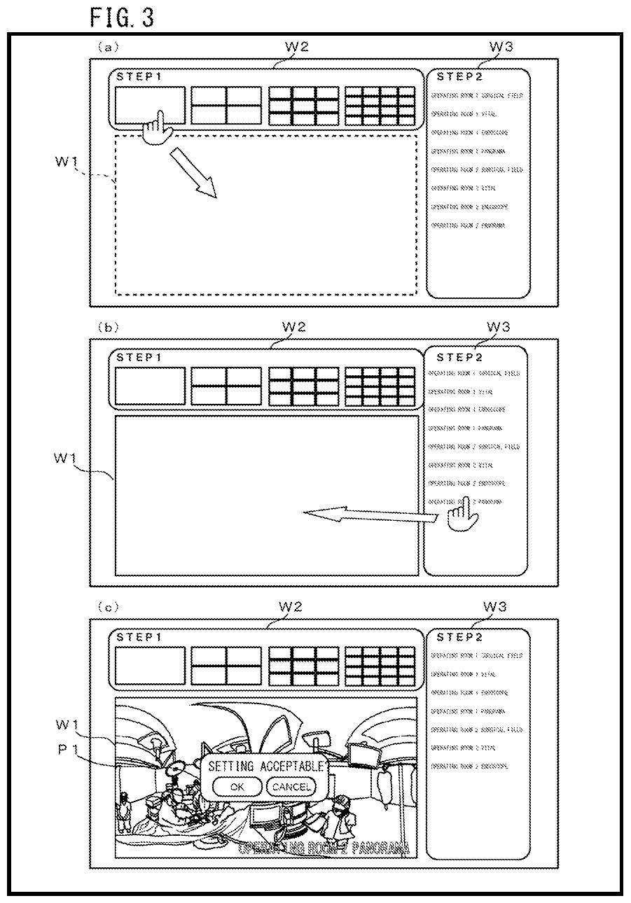

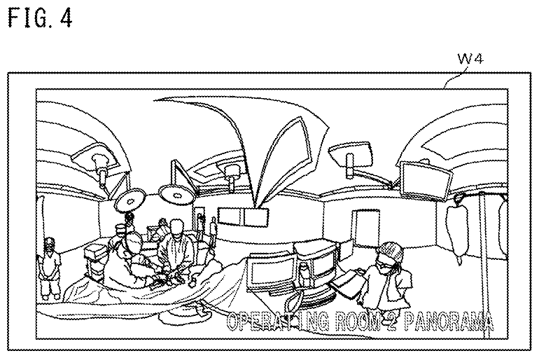

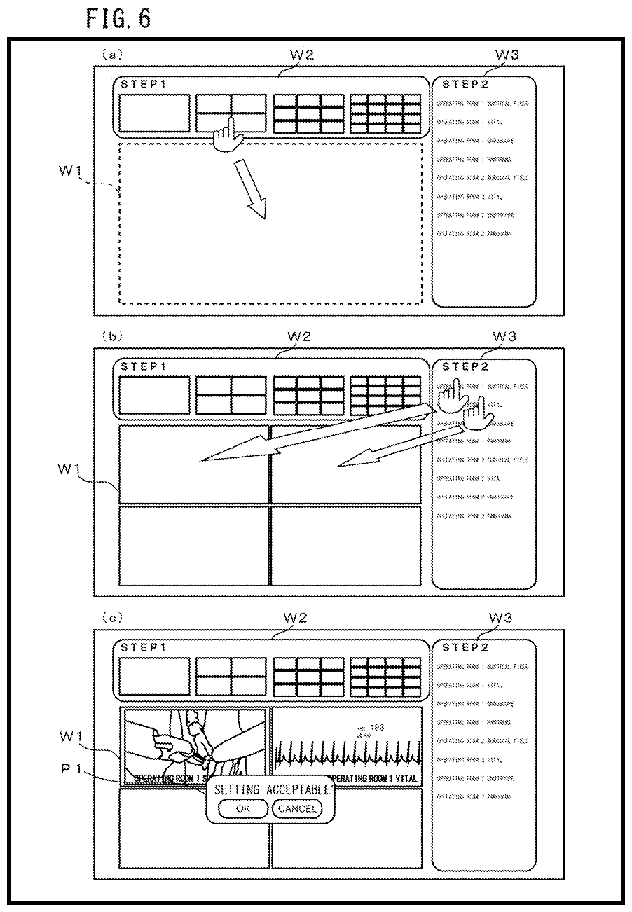

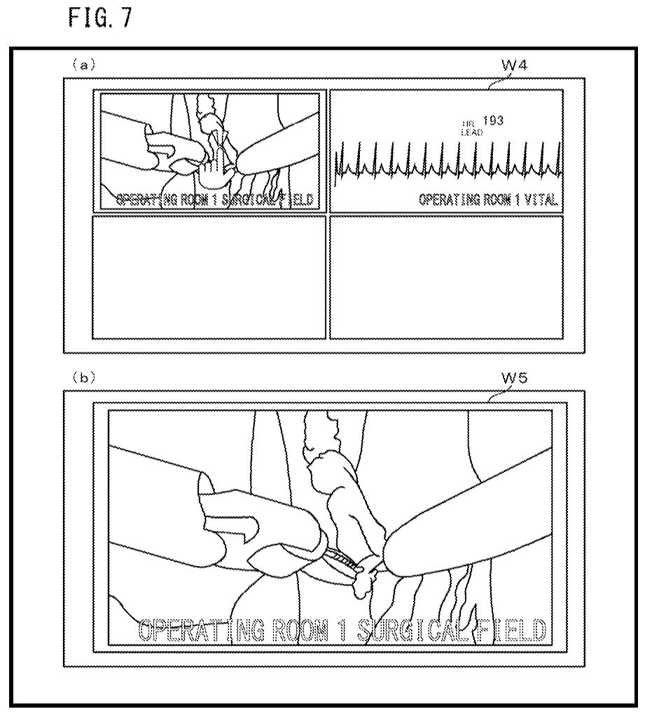

FIGS. 3 and 6 are drawings illustrating operation methods and screen shots of the display terminal 151 in layout designation and correspondence designation. FIG. 4 is a drawing illustrating a screen shot of a video streamed on the display terminal 151. FIG. 7 is a drawing illustrating screen shots of the videos streamed on the display terminal 151 and operation methods on the display terminal 151.

First, action procedures, by which a single video data will be displayed in a display area (a display window W4 illustrated in FIG. 4) of the display terminal 151, will be explained.

The display terminal 151 executes the origin designation process designating one or a plurality of origins of the video data from among the plurality of medical devices, and posts the designated origin(s) to the server device 160 (step S101). The origin designation process may be conducted, when for example there is a single video data output from the medical device, so as to designate such medical device per se, meanwhile when there are a plurality of output channels owned by the medical devices (when there are a plurality of video data output from the medical devices), the origin may only be designated for each of the output channels.

While there is no special limitation on technique of designating the medical device which serves as the origin in the origin designation process, this embodiment employs, as mentioned previously, the IP address having been assigned to such medical device(s) or to the IP converter(s) connected to such medical device(s).

Note that, while the number of origins designatable by the origin designation process is not specifically limited, this embodiment will be explained below presupposing that all of those aforementioned as being equivalence of the medical devices (the surgical field cameras 110 and 115, the biomonitors 120 and 125, the endoscope cameras 130 and 135, and the panorama cameras 140 and 145) are designated.

Note also that, the display terminal 151 explained below in this embodiment is understood to individually add, as the identification information, “operating room 1 surgical field” to the surgical field camera 110 designated in the origin designation process, “operating room 2 surgical field” to the surgical field camera 115, “operating room 1 vital” to the biomonitor 120, “operating room 2 vital” to the biomonitor 125, “operating room 1 endoscope” to the endoscope camera 130, “operating room 2 endoscope” to the endoscope camera 135, “operating room 1 panorama” to the panorama camera 140, and “operating room 2 panorama” to the panorama camera 145.

These identification information in this process may be freely determined in response to an user's operation, or may be determined by the display terminal 151 according to an automatic process (for example, a process for referring to a table used for converting the IP address to the correspondent identification information). Alternatively, the process for determining the identification information is not always necessarily conducted by the display terminal 151, but may be conducted for example by the server device 160.

The server device 160 checks whether the video data from the medical devices that fall under the origins posted by the display terminal 151 in step S101 are stored or not, and returns the result to the display terminal 151 (step S102).

Note that in such response in step S102, the server device 160 is understood to respond to the display terminal 151 that the server device 160 stores video data from the medical devices that correspond to all origins having been posted (response to the post in step S101 enabled).

When the video data from the medical devices were not stored for a part of, or all of the origins having been posted, the server device 160 posts the fact (response to the post in step S101 disabled) to the display terminal 151.

The display terminal 151 conducts the layout designation process (step S103) for designating a layout of the video data to be displayed on the display terminal, and the server device 160 makes a response stating that the layout designated in step S103 has been accepted (step S104).

The display terminal 151 then conducts the correspondence designation process that freely designates to which display areas the origins having been designated by the origin designation process to be correlated, out of one or a plurality of display areas contained in the layout designated by the layout designation process, in response to a user's operation (step S105).

Methods for specific operations by the user regarding the processes from step S103 to step S105 will be explained below.

The display terminal 151 displays a main window W1 which is a major part involved in layout designation and correspondence designation. The display terminal 151 also displays a layout selection window W2 that contains icons individually representing layout choices (layout with a single display area, 4-split layout, 9-split layout, 16-split layout). The display terminal 151 also displays an origin selection window W3 that contains the origins designated by the origin designation process.

Assume now that the icon representing the layout with a single display area (the leftmost icon), out of four icons displayed in the layout selection window W2, was chosen through a pointing operation by the user, and that the icon was dragged and dropped into the main window W1 (see FIG. 3(a)).

This operation designates the layout with a single display area, and the layout will appear in the main window W1 of the display terminal 151.

Assume now that “operating room 2 panorama” that corresponds to the panorama camera 145, from among the identification information contained in the origin selection window W3, was chosen through a pointing operation by the user, and then dragged and dropped into the main window (see FIG. 3(b)).

This operation thus correlates the display area contained in the layout, with the panorama camera 145 assumed as the origin.

As described above, the layout designation process in this embodiment is featured by that the number of designatable display areas contained in the layout (any of one, four, nine and sixteen) does not depend on the number of medical devices designated by the origin designation process. This successfully makes the correspondence designation process more flexible, and makes the layout more user-friendly.

In some cases, the number of display areas contained in the layout, designatable by the layout designation process, can be larger than the number of origins designated by the origin designation process. A possible layout in such case may be given typically by dividing, in the correspondence designation process, the display area for displaying the video data for each operating room.

The server device 160 edits the video data output from the origin designated in step S101, in the display area contained in the layout designated in step S103, referring to the correspondence designated in step S105 (step S106).

More specifically, the server device 160 conducts the editing process which is a process for adding an indication that represents the identification information named “operating room 2 panorama” designated to the panorama camera 145 as the origin, to the display area contained in the layout.

The editing process in this context may only assign the video data output from the designated origin to the display area contained in the designated layout, referring to the designated correspondence, and does not always necessarily contain a process for attaching an indication that represents the identification information to a target video data.

Note that the aforementioned editing process (process in step S106) is denoted as “Editing (unsynthesized)” in FIG. 2, since the process is not intended to produce synthesized video data described later.

The server device 160 then distributes the video data having gone through the editing process, as an image video having been defined by the foregoing designation, to the display terminal 151 (step S107).

The display terminal 151 displays in the main window W1 the video data distributed in step S107, and overlays on the display a pop-up display P1 that prompts the user to choose whether or not the current designation is acceptable (“OK” or “CANCEL”) (see FIG. 3(c)).

If the user points on “OK”, acceptance of the designation is posted to the server device 160 (step S108), and the video data having been appearing on the main window W1 is streamed from the server device 160 to the display terminal 151 and displayed in the display window W4 (step S109, see FIG. 4). The display window W4 in this case is enlarged beyond the main window W1, since the display areas for the layout selection window W2 and the origin selection window W3 are omitted.

If the user points on “CANCEL”, designations on the layout designation and the correspondence designation are judged to be ignored, and the process restarts from step S103.

As a result of the aforementioned action procedures between the display terminal 151 and the server device 160, the display terminal 151 will have the video data from the panorama camera 145 displayed in the display area (display window W4), with the identification information corresponding to the panorama camera 145 attached thereto.

Next paragraphs will explain the action procedures by which a plurality of video data will be displayed in the display area of the display terminal 151 (display window W4 illustrated in FIG. 7(a)), and then a single video data will be displayed in the display area of the display terminal 151 (display window W5 illustrated in FIG. 7(b)).

The display terminal 151 conducts the origin designation process by which one or a plurality of origins of the video data are designated from among the plurality of medical devices, and posts the designated origin(s) to the server device 160 (step S201).

The server device 160 checks whether the video data from the medical devices that fall under the origins posted by the display terminal 151 in step S201 are stored or not, and returns the result to the display terminal 151 (step S202).

Note that the processes in step S201 and step S202 are substantially same as the aforementioned processes in step S101 and step S102, and will not be detailed again.

The display terminal 151 conducts the layout designation process for designating a layout of the video data to be displayed on the display terminal (step S203). The server device 160 makes a response stating that the layout designated in step S203 has been accepted (step S204).

The display terminal 151 then conducts the correspondence designation process that freely designates to which display areas the origins having been designated by the origin designation process to be correlated, out of one or a plurality of display areas contained in the layout designated by the layout designation process, in response to a user's operation (step S205).

Methods for specific operations by the user regarding the processes from step S203 to step S205 will be explained below.

The display terminal 151 displays the main window W1, the layout selection window W2, and the origin selection window W3. Assume now that the icon representing the 4-split layout of the display area (the second leftmost icon), out of four icons displayed in the layout selection window W2, was chosen through a pointing operation by the user, and that the icon was dragged and dropped into the main window W1 (see FIG. 6(a)).

This operation designates the layout representing a 4-split display area, and such layout will appear in the main window W1 of the display terminal 151.

Assume now that, in response to pointing operations made by the user, and from among the identification information contained in the origin selection window W3, “operating room 1 surgical field” that corresponds to the surgical field camera 110 was chosen and then dragged and dropped into the display area positioned in the upper left of the main window W1, and that “operating room 1 vital” that corresponds to the biomonitor 120 was chosen and then dragged and dropped into the display area positioned in the upper right of the main window W1 (see FIG. 6(b)).

This operation thus correlates the upper left display area and the upper right display area contained in the layout, respectively with the surgical field camera 110 and the biomonitor 120 assumed as the origins.

The server device 160 edits the video data output from the origin designated in step S201, in the display area contained in the layout designated in step S203, referring to the correspondence designated in step S205. (step S206)

More specifically, the server device 160 in the editing process synthesizes the plurality of video data output from the origins, to thereby produce a single synthesized video data having a data volume nearly equal to that of the video data output from a single origin. The server device 160 further adds an indication that represents an identification information named “operating room 1 surgical field” specified to the surgical field camera 110, and an indication that represents an identification information named “operating room 1 vital” specified to the biomonitor 120, respectively to the display areas (the upper left display area and the upper right display area) contained in the layout designated by the playback request.

Note that this editing process is denoted as “Editing (synthesized)” in FIG. 6, since the process is intended to produce synthesized video data.

The server device 160 then distributes the video data having gone through the editing process in step S206 (a single synthesized video data), as an image video having been defined by the foregoing designation, to the display terminal 151 (step S207).

The display terminal 151 displays in the main window W1 the video data distributed in step S207, and overlays on the display the pop-up display P1 that prompts the user to choose whether or not the current designation is acceptable (“OK” or “CANCEL”) (see FIG. 6(c)).

If the user points on “OK”, the designation is accepted (step S208), and the video data having been appearing on the main window W1 is streamed from the server device 160 to the display terminal 151 and displayed in the display window W4 (step S209, see FIG. 7(a)). The display window W4 in this case is enlarged beyond the main window W1, since the display areas for the layout selection window W2 and the origin selection window W3 are omitted.

If the user points on “CANCEL”, designations on the layout designation and the correspondence designation are judged to be ignored, and the process restarts from step S203.

Unlike the streaming in step S109, the streaming in step S209 allows the server device 160 to synthesize a plurality of video data to produce a single synthesized video data, and streams it towards the display terminal 151. Note that the streaming in step S209 corresponds to the first distribution process in the context of the present invention.

Also note that, as described previously, the video data (a single synthesized video data) streamed in step S209 has a data volume nearly equal to that of the video data output from a single origin. Hence, the traffic in a case where a single origin is designated and streamed (that is, streaming in step S109) will become nearly equal to the traffic of the streaming in step S209, making it possible to stabilize the traffic of the medical video distribution system 100 as a whole.

Now the “nearly equal data volume” means that the difference between both video data volumes to be compared falls within a range two or more digits smaller (<+10%) than the data volume of the whole video data.

In a case where a plurality of display areas are contained in the layout of the video data being reproduced on the display terminal 151, such as during the streaming in step S209, the user can freely designate one display area from the layout, by repeating the pointing operation twice (double clicking) (see FIG. 7(a)).

For example, in a case where the upper left display area in the 4-split layout was designated (step S210), the server device 160 reads the video data output from the origin corresponded to the display area freely designated by the user, and streams the video data towards the display terminal 151 (step S211). Note that the streaming in step S211 corresponds to the second distribution process in the context of the present invention.

The video data (a single video data) streamed in step S209 has, as described previously, the data volume nearly equal to that of the video data output from a single origin. Moreover, the streaming in step S211 is intended to stream the video data from a single origin. Hence, the traffic of the streaming in step S211 (second distribution process) becomes nearly equal to the traffic of the streaming in step S209 (first distribution process), making it possible to stabilize the traffic of the medical video distribution system 100 as a whole.

Now “traffic” means amount of information (communication volume) on a communication line of the medical video distribution system 100.

Moreover, the “nearly equal traffic” means that the difference between both traffics to be compared, presupposing that their traffics are in stable condition, falls within a range two or more digits smaller (<±10%) than the whole traffic.

As a result of the aforementioned action procedures between the display terminal 151 and the server device 160, a plurality of video data will be displayed in the display area (display window W4) of the display terminal 151, and then a single video data will be displayed in the display area (display window W5) of the display terminal 151.

It is now understood that, in the action procedures illustrated in FIG. 5, step S201 corresponds to the first step of the present invention, step S203 corresponds to the second step of the present invention, step S205 corresponds to the third step of the present invention, step S206 corresponds to the fourth step of the present invention, and step S209 corresponds to the fifth step of the present invention.

It is also understood that, in the action procedures illustrated in FIG. 5, step S210 corresponds to the sixth step of the present invention, and step S211 corresponds to the seventh step of the present invention.

In other words, FIG. 5 illustrates the medical video distribution method of the present invention.

### Modified Example of Present Invention

Having described the present invention on the basis of embodiments explained referring to the individual drawings, the present invention is not limited to the aforementioned embodiment, instead allowing various modifications and improvements so long as the objects of the present invention can be achieved.

Although having explained the foregoing embodiments on the premise of constituents illustrated in FIG. 1, the individual constituents of the present invention may only be formed enough to embody their functions. Hence, the individual constituents of the present invention are not always necessarily independent from each other, instead allowing that a plurality of constituents are formed into a single member, that a single constituent is composed of a plurality of members, that a certain constituent is a part of other constituent, and that a part of certain constituent overlaps a part of other constituent.

For example, although having illustrated the medical video distribution system of the present invention with four display terminals, a fewer number of display terminals (including a single display terminal) may be contained, or a larger number of display terminals may be contained.

Alternatively, although having illustrated the medical video distribution system of the present invention that contains medical device groups installed in two operating rooms, the system may only contain the medical device group installed in a single operating room, or contain the medical device groups installed in a larger number of operating rooms.

Although having explained the foregoing embodiments in which the number of display areas contained in the layout designatable in the layout designation process becomes larger than the number of origins designated in the origin designation process, the relationship may be inverted. That is, the number of display area contained in the layout designatable in the layout designation process may become smaller than the number of origins designated in the origin designation process.

The various operations described in the foregoing embodiments are merely exemplary ones, by which the present invention is by no means limited. Hence the foregoing individual operations may be replaced by other operations, may be partially omitted, or other operations having not been explained in the embodiments may be added.

The various screen shots described in the foregoing embodiments are merely exemplary ones, by which the present invention is by no means limited. Hence the foregoing individual screen shots may be replaced by other screen shots, may be partially omitted, or other screen shots having not been explained in the embodiments may be added.

This embodiment encompasses technical spirits below.

(1) A medical video distribution system that includes: a plurality of medical devices that output video data; a server device that stores the video data output from the medical devices; and one or a plurality of display terminals that receive, and then play, the video data received from the sever device by way of streaming, the display terminal being designed to execute: an origin designation process designating one or a plurality of origins of the video data from among the plurality of medical devices; a layout designation process designating a layout of the video data to be displayed on the display terminal(s); and an correspondence designation process freely designating to which display area(s) the origin(s) having been designated by the origin designation process to be correlated, out of one or a plurality of display areas contained in the layout designated by the layout designation process, in response to an user's operation, and, the server device being designed to execute: an editing process editing the video data output from the origin(s) designated by the origin designation process, on a basis of the correspondence designated by the correspondence designation process, in the display area(s) contained in the layout designated by the layout designation process; and a first distribution process streaming the video data having been edited by the editing process towards the display terminal(s), and, when there are plurality of origins designated by the origin designation process, the server device being designed to synthesize, in the editing process, a plurality of video data output from the origins to produce a single synthesized video data having a data volume nearly equal to that of the video data output from a single origin, and to stream, in the first distribution process, the single synthesized video data towards the display terminal(s).

(2) The medical video distribution system according to (1), wherein, when there are the plurality of display areas contained in the layout of the video data being played on the display terminal(s), and the user freely specified one display area from the layout, the server device executes a second distribution process reading the video data output from the origins correlated to the display area(s) freely specified by the user, and streaming the video data towards the display terminal(s), and wherein, the second distribution process has a traffic of the streaming, nearly equal to a traffic of the streaming in the first distribution process.

(3) The medical video distribution system according to (1) or (2), wherein the server device adds, in the editing process, an indication that represents an identification information specified to each origin by the origin designation process, to each of the display areas contained in the layout specified by the layout designation process.

(4) The medical video distribution system according to any one of (1) to (3), wherein the number of the display area(s) contained in the layout, designatable by the layout designation process, does not depend on the number of medical device(s) designated by the origin designation process.

(5) The medical video distribution system according to (4), wherein the number of the display area(s) contained in the layout, designatable by the layout designation process, can exceed the number of the medical devices designated by the origin designation process.

(6) A medical video distribution method using: a plurality of medical devices that output video data; a server device that stores the video data output from the medical devices; and one or a plurality of display terminals that receive, and then play, the video data received from the sever device by way of streaming, the method includes: a first step designating one or a plurality of origins of the video data from among the plurality of medical devices; a second step designating a layout of the video data to be displayed on the display terminal(s); a third step freely designating to which display area(s) the origin(s) having been designated by the first step to be correlated, out of one or a plurality of display areas contained in the layout designated by the second step, in response to an user's operation; a fourth step editing the video data output from the origin(s) designated by the first step, on a basis of the correspondence designated by the third step, in the display area(s) contained in the layout designated by the second step; and a fifth step streaming the video data having been edited by the fourth step towards the display terminal(s), the method being designed, when there are plurality of origins designated by the first step, to synthesize in the fourth step a plurality of video data output from the origins to produce a single synthesized video data having a data volume nearly equal to that of the video data output from a single origin, and to stream, in the fifth step, the single synthesized video data towards the display terminal(s).

(7) The medical video distribution method according to (6), further includes: a sixth step allowing the user to freely specify, when there are the plurality of display areas contained in the layout of the video data being played on the display terminal, one display area from the layout; and a seventh step reading the video data output from the origin correlated to the display area freely specified by the user in the sixth step, and streaming the video data towards the display terminal, and wherein, the seventh step has a traffic of the streaming, nearly equal to a traffic of the streaming in the fifth step.

This application claims priority to Japanese Patent Application No. 2018-235455 filed on Dec. 17, 2018, the entire contents of which are incorporated by reference herein.
100 medical video distribution system
110, 115 camera
111, 116, 121, 126, 131, 136 IP converter
120, 125 biomonitor
130, 135 endoscope camera
140, 145 panorama camera
151, 152, 153, 154 display terminal
160 server device
W1 main window
W2 layout selection window
W3 origin selection window
W4 display window
W5 display window

1-7. (canceled)

8. A medical video distribution system comprising:
a plurality of medical devices that output video data;
a server device that stores the video data output from the medical devices; and one or a plurality of display terminals that receive, and then play, the video data received from the sever device by way of streaming,
the display terminal being designed to execute;
an origin designation process designating one or a plurality of origins of the video data from among the plurality of medical devices;
a layout designation process designating a layout of the video data to be displayed on the display terminal(s); and
an correspondence designation process freely designating to which display area(s) the origin(s) having been designated by the origin designation process to be correlated, out of one or a plurality of display areas contained in the layout designated by the layout designation process, in response to an user's operation,
and,
the server device being designed to execute:
an editing process editing the video data output from the origin(s) designated by the origin designation process, on a basis of the correspondence designated by the correspondence designation process, in the display area(s) contained in the layout designated by the layout designation process; and
a first distribution process streaming the video data having been edited by the editing process towards the display terminal(s),
and,
when there is a single origin designated by the origin designation process, the server device assigns, in the editing process, a single video data output from the designated origin to the display area contained in the designated layout, and streams in the first distribution process the single video data towards the display terminal,
and,
when there are plurality of origins designated by the origin designation process, the server device synthesizes, in the editing process, a plurality of video data output from the origins to produce a single synthesized video data having a data volume nearly equal to that of the single video data streamed when there were a single origin designated by the origin designation process, and streams in the first distribution process the single synthesized video data towards the display terminal(s).

9. The medical video distribution system according to claim 8, wherein,
when the user freely designated a single display area in the layout of the single synthesized video data being played on the display terminal(s), the server device executes a second distribution process reading the video data output from the origins correlated to the display area(s) freely specified by the user, and streaming the video data towards the display terminal(s),
and wherein,
the second distribution process has a traffic of the streaming, nearly equal to a traffic of the streaming in the first distribution process.

10. The medical video distribution system according to claim 9, wherein the server device adds, in the editing process, an indication that represents an identification information specified to each origin by the origin designation process, to each of the display areas contained in the layout specified by the layout designation process.

11. The medical video distribution system according to claim 8, wherein the number of the display area(s) contained in the layout, designatable by the layout designation process, does not depend on the number of medical device(s) designated by the origin designation process.

12. The medical video distribution system according to claim 11, wherein the number of the display area(s) contained in the layout, designatable by the layout designation process, can exceed the number of the medical devices designated by the origin designation process.

13. A medical video distribution method using:
a plurality of medical devices that output video data;
a server device that stores the video data output from the medical devices; and
one or a plurality of display terminals that receive, and then play, the video data received from the sever device by way of streaming,
the display terminal executing:
a first step designating one or a plurality of origins of the video data from among the plurality of medical devices;
a second step designating a layout of the video data to be displayed on the display terminal(s); and
a third step freely designating to which display area(s) the origin(s) having been designated by the first step to be correlated, out of one or a plurality of display areas contained in the layout designated by the second step, in response to an user's operation,
and,
the server device executing:
a fourth step editing the video data output from the origin(s) designated by the first step, on a basis of the correspondence designated by the third step, in the display area(s) contained in the layout designated by the second step; and
a fifth step streaming the video data having been edited by the fourth step towards the display terminal(s), and
when there is a single origin designated by the first step, the server device assigns, in the fourth step, a single video data output from the designated origin to the display area contained in the designated layout, and streams in the fifth step the single video data towards the display terminal, and,
when there are plurality of origins designated by the first step, the server device synthesizes, in the fourth step, a plurality of video data output from the origins to produce a single synthesized video data having a data volume nearly equal to that of the single video data streamed when there were a single origin designated by the first step, and streams in the fifth step the single synthesized video data towards the display terminal(s).

14. The medical video distribution method according to claim 13, further comprising:
a sixth step posting the single display area freely designated by the user in the layout of the single synthesized video data being played on the display terminal, from the display device to the server device, and
a seventh step allowing the server device to read the video data output from the origin correlated to the display area freely specified by the user in the sixth step, and to stream the video data towards the display terminal,
and wherein,
the seventh step has a traffic of the streaming, nearly equal to a traffic of the streaming in the fifth step.
